(19) United States
(12) Patent Application Publication
(10) Pub. No.: US 2009/0299389 A1
(43) Pub. Date: Dec. 3, 2009

(54) FLANGELESS PROSTHESIS FOR ANASTOMOSIS

(76) Inventor: Luiz Gonzaga Granja Filho, Recife/PE (BR)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 12/303,541

(22) PCT Filed: Jun. 6, 2007
(86) PCT No.: PCT/BR2007/000142

§ 371 Date: Jul. 8, 2009

(30) Foreign Application Priority Data
Jun. 6, 2006 (BR) PI 0602378-9

(51) Int. Cl. A61B 17/11; A61F 2/06

(52) U.S. Cl. 606/155; 623/1.36

(57) ABSTRACT

A prosthetic device is provided that is used in end-to-side, end-to-end, and side-to-side anastomosis without clamping and sutureless, or with expeditious clamping and sutureless, wide, single or multiple, at the same time, without the risk of kinking, where the graft is inserted in at least one of the intraluminal portions of the prosthesis tubular member. The tubular member is flangeless and comprises double holes or small external handles in its extension where wires pass to fix the prosthesis to the organ walls and/or the grafts to prosthesis.

### FIELD OF INVENTION

The present invention relates in a general manner to anastomotic devices and more specifically to a flangeless prosthetic device that allows anastomosis without clamping and sutureless, or with expeditious clamping and sutureless (in normal wall organs, not friable or calcified), wide, single or multiple, at a single time, where a vascular graft or anastomotic trunk (joint of many grafts by suture or any method), or any other graft, is inserted in the lumen of the prosthesis and reversed by jacketing to cover part of the prosthesis that will remain inside the graft (vein, artery or any biological or synthetic tissue), and is fixed to side wall of the tubular portion by double holes, or small handles, directly and/or by circumferential ligature of grafts to the prosthesis, preferably. In short prosthesis and thick gauge, the graft or anastomotic trunk can also cover only the externally the prosthesis upper end, without coating it internally. The prosthesis comprises a plurality of openings, or small external handles, equidistant, placed throughout the tubular member, allowing it to be sutured on tissue, vein, artery or any other organ, out of anastomosis, eliminating one of the main causes of stenosis and/or obstruction of anastomosis, which is the introduction of foreign bodies inside the lumen, due to the reaction of foreign body that occurs in anastomosis site and also eliminating the clamping, which is the main factor that is responsible for thromboembolism and tissue lacerations, especially of friable and/or calcified structures.

### DESCRIPTION OF THE PRIOR ART

A prior art presents several trials provide solutions for anastomotic devices projected to correct vascular abnormalities, which present the following typical features:

The North-American U.S. Pat. No. 3,254,650, of Jun. 7, 1966, describes a method and devices to execute anastomosis procedures by applying with adhesive two separated connectors in a body member and removing this body member portion contained among the connectors, joining the said connection devices for joining the remaining portions of the body member.

The U.S. Pat. No. 3,265,069, of Aug. 9, 1966, describes devices or instruments for use by surgeons in reunion of body ducts, which in the course of operations were separated. The instruments comprise a pair of elongated similar elements and articulately connected, in an intermediary manner, and with an support for finger retention in a distal end, comprising a generally cylindrical shape with a cylindrical channel that passes through it in the other distal end, in order to receive tubular body ducts kept by the instrument while the body ducts are reconnected.

U.S. Pat. No. 3,774,615, of Nov. 27, 1973, describes a device to connect the end of interrupted tubular organs without sewing, comprising a connecting ring on which the end of the interrupted organ are pulled, the ring is preferably locked up by a fixation resource. The ring and fixation resource are made of inert material, and preferably a hydrophile gel that can be dilated until its equilibrium or can be a hydrogel incompletely dilated, which is submitted to additional dilatation where it is applied. The connecting ring can be supplied with a groove and can be placed in a ring shaped fixation resource and kept there joining it to the fixation resource in the groove or simply kept by a screw. Two connection rings can also be used and kept joined by a coupling member.

The document U.S. Pat. No. 4,366,819, of Jan. 4, 1983, describes an anastomotic joint for surgery with a graft of coronary artery deviance comprising a mounting of four elements including a cylindrical tube with at least one locking indentation of ring flange in one influx end and a plurality of grooves of locking ring in a flow end; a ring flange with a central opening and a plurality of long and short spigots, the long spigots are engaged in the locking indentation, with a graft engaged among them; a fixation ring with a central opening and a plurality of spigots positioned around the opening; and a locking ring with a opening with a plurality of locking ring edges for engaging with the locking ring grooves. In surgical implants, an aortic wall with a hole engages between the ring flange and the fixation ring and is kept in this position by spigots of the fixation ring, and the four elements engage together forming an integral anastomotic joint. A first alternate modality includes an anastomotic joint of three elements with a combination of fixation ring and locking ring. A second alternate modality includes an anastomotic joint of four elements with a slightly jolted end in an influx end, exposing the graft material in the anastomotic “ostium”.

Other prior arts are equally mentioned, base don some information of “The Cardiothoracic Surgery Network”. The “Simmetry Aortic Connector System”, developed by St. Jude Medical, is a connector made with nitinol, selected by vein diameter with an adventicia removed to allow adjust of the connector and to prevent its displacement by the blood current. Then, the device may make an angle of 90° with the aorta. Among the disadvantages, there is the fact that it can be used only in extreme cases due to the difficult usage of this technique; it did not obtain a satisfactory result in many surgeries and it is being drowned out of market by the manufacturer; it is not applicable in calcified aorta; presents suture; presents contact with blood flow (foreign body); it does not widen the anastomosis area (restrictive anastomosis); performs only one anastomosis at a time; it is a product restrict to end-to-side anastomosis; a great mobilization of the venous graft occurs, damaging it, and can eventually form thrombus; there is a risk of perforation of the posterior wall of aorta; and the adventicia is removed (most resistant vascular layer).

Other known device is the PAS-Port™System, a device used in 3 steps, and the vein wall is mounted over the device and is manually reversed on it, by tool and adapted to aorta with an angle of 90°. The method alerts that the surgeon shall select with due care the point of aorta and the vein size. The device is made of stainless steel and is available in only one size that allows the use of veins with external diameter of 4 to 6 mm, aorta with an internal diameter of 18 mm. It is available in only one size, limiting its applicability. As disadvantages of this prior art, the device has contact with blood flow (foreign body); it does not widen the anastomosis area (restrictive anastomosis); it uses veins with external diameter of 4 to 6 mm and aorta with an internal diameter of 18 mm; it does not perform multiple nor visceral anastomosis; it performs just only end-to-side anastomosis; a great mobilization of the used biological graft occurs, damaging its inner layer, which generates the formation of thrombus; there is a big risk of kinking at the origin (angle of 90°) and risk of posterior wall perforation in the aorta at the moment the device is introduced under its light; the suture is substituted with disadvantages by stainless steel (9 pins, distant among them, maximizing the risk of bleeding).

Also as prior art, there is the CorLink Device, currently commercialized by Ethicon/Johnson & Johnson, that allows the creation of anastomosis between the ascending aorta and a saphenous vein segment. Aortic Anastomotic Device (AAD) is a self-expanded device with extra luminal nitinol constituted by a central cylinder with five interconnected elliptical archs and 2 groups of 5 pins in the end portion of the cylinder. The pins, after the eversion of venous walls in the device, fix the aggregate penetrating into the venous graft wall. A blade makes an opening in the wall of aorta and permits the coupling of AAD, which also fix the wall of aorta by pins. With this device: it poses a serious risk of bleeding, especially in friable aortas, thin, calcified or fibrous, restricting its applicability, also with risks, even in aortas with normal walls; in small gauge anastomosis, there is a risk of thrombosis, hyperplasia, intimal proliferation and fibrosis (reaction to foreign body type in origin of anastomosis) with consequent stenosis resulting in occlusion of anastomosis; sutures are used in some cases; there is cases of infarction caused by equipment; there is a recurring need of re-operations in patients; the device presents contact with blood flow (foreign body); it is not flexible; it does not multiple anastomosis; an inadequate mobilization of venous graft occurs, and can cause damage to its intimal layer, it could form thrombus; it is used only in extreme cases because it is a technique of complex usage; the suture is substituted by stainless steel in contact with blood flow.

Another known device is the St Jude Distal Connector that consists of a stainless steel clip mounted on a catheter, comprising a balloon for subsequent expansion and connector mounting. The catheter is introduced backward from the end, by doing a small hole in the anastomosis site, the clip fixes the vein in the hole, the catheter goes to coronary and releases the connector. The catheter is removed and a suture is done in side-to-side anastomosis. With St Jude Distal Connector, occurrence of leakage problems were detected in 20% of the used connectors; the use of a metallic clip requires due care for handling to avoid distortion in the anastomosis; late angyographies reveal smaller circular diameter of anastomosis made with o St. Jude Distal Connector, when compared to controls made with conventional suture; there is remarkably risk of bleeding and the graft is very mobilized, and lacerations can occur in its inner layer, allowing the formation of thrombus.

The HeartFlo™ is a multi-suture instrument for anastomosis with wires automatically applied in end-to-side and side-to-side anastomosis. The surgeon manually ties the suture wires (10 wires) and concludes the anastomosis similarly to the traditional process. Besides of being a product of complex handling, it makes suture in anastomosis (keeping the undesirable foreign body in the internal origin of the anastomosis) and is restricted to end-to-side and side-to-side anastomosis. There is also an excessive mobilization of graft, and can cause lesions in its intimal layer, which would be the inductor that forms the thrombus.

Another technique and known device is the Solem Graftconnector, produced by the swedish company Jomed. It is constituted by a stent made of nickel and titanium coated with polytetrafluorethylen used to connect the internal thoracic artery the left anterior descending coronary artery. The results has not been satisfactory, because it poses risk of bleeding; there is also an excessive mobilization of graft, probably damaging intimal layers, allowing the formation of thrombus; it is not flexible, by this fact, causes trauma to grafts; it does not make multiple anastomosis, at a single time; presents contact with blood flow (foreign body); and is frequent the need of-operations.

The Magnetic Vascular Positioner System is produced by Ventrica and comprises 4 magnetic rings and the anastomosis is processed by magnetic attraction of 4 ports. However, initial experimental results demonstrate leakage, also an undesired contact of materials with blood flow. On the other hand, it is necessary to be careful to avoid the capitation of excess of tissue among the magnets. With this system, there is also a need of suture in some cases; there is occurrence of infarction caused by equipment; and is frequent the need of-operations in patients; and also requires clamping.

Also, as a device known by the medical area, the Combined Anastomotic Device and Tissue Adhesive, developed by Grundeman & Borst group, combines micro mechanical technique with use of adhesive (glue). The use of this method can result in leakages and need traditional sutures; it is frequent the need of re-operation due to leakage/bleeding; and performs only one anastomosis at a time.

Finally, it is also experimentally practiced anastomosis assisted by laser, where the results are not different from conventional isolated sutures, because there is a need of suture in some cases; there is a risk of bleeding e leakage; and does not perform multiple anastomosis.

Even so divulged nowadays, anastomosis with clamper, by insecurity, and almost totality of surgeons perform conventional sutures throughout the route of anastomosis, with an intention of avoiding leakages and bleedings, it means the use of dampers just makes the procedure more expensive, once the conventional suture is also applied.

In short, the conventional anastomosis, with clamping and with suture, standardized in 1906 by Aléxis Carrel, remains the first choice for any type of anastomosis and organs to be anastomosed.

With an expectation of changing the current situation, the Brazilian patent n°. PI 9706197-2, describes and claims a prosthesis for vascular anastomosis or in any other organ or tissue, without the use of clamping and sutureless, solving, in an elegant and efficient manner, the limitations inherent to prosthesis of the above mentioned prior art, when used in vascular anastomosis performed, mainly in thin aortas, calcified and friable; or in any other application where a clamping of a vein or artery can pose excessive trauma for conditions of a given patient. The prosthesis that is subject of that request allows the embodiment of fast and safe anastomosis, without obstruction of vein or artery lumen of which anastomosis is made, also allows anastomosis in tissues, veins or arteries in bad conditions and never would accept a clamping used in conventional anastomosis. This is achieved by a generally cylindrical shaped prosthesis with a flange orthogonally extending from its external side wall, in a point in the prosthesis length between its ends, the referred flange has openings distributed around its surface. The description of the usage method and specific construction of the prosthesis is presented in the drawings of the descriptive report of that request, as well as the document C19706197-2, Certificate of Addition of the first.

Although these anastomotic devices can be presented as suited to the purposes for which they were projected, they are not so suited for the purposes of the present invention, as described herein below.

### SUMMARY OF THE INVENTION

The present invention relates to variations of currently known anastomotic devices, in order to allow side-to-side, end-to-end and end-to-side anastomosis without clamping and sutureless, or with expeditious clamping and sutureless, in organs with normal walls, where at least one vascular graft, or any other, is inserted into the prosthesis lumen and reversed by jacketing to cover partially the prosthesis, and is fixed in the side wall of tubular portion by double holes or circumferential, small external handles, equidistant, or also circumferential ligature of grafts to external surface of tubular member, with grooves. The double holes or small external handles, equidistant, longitudinally aligned or not, placed throughout the tubular member, allow the prosthesis to be sutured in the tissue, vein, artery or any other organ, out of anastomosis, so eliminating one of the main causes of stenosis and/or obstruction of anastomosis which is the introduction of foreign bodies inside the lumen, due to a type of reaction to foreign body that occurs at the anastomosis site and also eliminating the clamping that is the main factor and responsible of thromboembolism and tissue lacerations, especially of friable structures and/or calcified. Other important characteristic is to avoid the grafts or trunk kinking, when they are fixed to the everted portion, distant from where they emerge in prosthesis, being free inside it. In short prosthesis and thick gauge, the graft or anastomotic trunk can also cover just the exterior of the prosthesis upper end, without coating it internally (here, the flowing fluid, ex. blood, has contact with the material of prosthesis). The prosthesis can also have varied sizes and shapes to accommodate simultaneously varied grafts sizes, number and types.

An objective of the present invention is to provide an anastomotic device comprised of a tubular member with external grooves and flangeless, allowing anastomosis of any type, in any number, at a single time, in tubular organs of any gauge and type.

Another objective of the present invention is to provide an anastomotic device that does not introduces any foreign body into or for anastomosis grafts.

Another objective of the present invention is to provide an anastomotic device that allows the anastomosis preparation of any type, any two tubular organs, without clamping.

Another objective of the present invention is to provide an anastomotic device, that avoids the occurrence of graft kinking when emerging from prosthesis.

Another objective of the present invention is to provide an anastomotic device that avoids the occurrence of protuberance of anastomotic aggregate in the light of the organ that receives it.

Another objective of the present invention is to provide an anastomotic device, that allows the preparation of wide anastomosis, multiple, at a single time, with just only one prosthesis.

Another objective of the present invention is to provide an anastomotic device that allows the preparation of anastomosis in organs comprising peristaltism without risk of occurrence of intususception of grafts or organs, in the area of anastomosis.

Other features and additional objectives of the present invention will become apparent from the following descriptions. These features will be described on sufficiently detailed levels to allow the technicians of the subject matter to implement the invention. Also, it is understood that other features can be used and structural changes can be made without leaving the scope of the invention. In the accompanying drawings, like reference numbers indicate identical or like parts throughout the several views.

Therefore, the following detailed description should not be taken as limiting the scope of the present invention which is defined by the appended claims.

### BRIEF DESCRIPTION OF THE DRAWINGS

The present invention may be understood more completely by reference to the following description and appended drawings, supported by examples, in which:

FIG. 1 illustrates a perspective view of flangeless prosthesis, with transfixing double holes, equidistant among them, placed in the tubular member, located in the upper and extraluminal ends.

FIG. 1 A illustrates a prosthesis that differs from prosthesis of FIG. 1 only by substituting the double holes by small external handles, also located in the upper and extraluminal ends of the tubular member.

FIG. 2 illustrates a perspective view of flangeless prosthesis, with double holes in same direction of the tubular portion, located in the upper and lower ends.

FIG. 2 A illustrates a prosthesis that differs from prosthesis of FIG. 2 by substituting the double holes by small external handles located in the upper and lower ends of the tubular member.

FIG. 3 illustrates a perspective view of the flangeless prosthesis, with double holes no same direction do tubular member, located in the upper, intermediary and lower ends of the tubular member.

FIG. 3 A illustrates a prosthesis that differs from prosthesis of FIG. 3 only by substituting the double holes by small external handles located in the upper, intermediary and lower ends of the tubular member.

FIG. 4 illustrates a perspective view of prosthesis for at least two grafts, flangeless, with double holes in the same direction of tubular portions, located in the upper, intermediary and lower ends of the tubular member.

FIG. 4 A illustrates a prosthesis in perspective that differs from prosthesis of FIG. 4 only by substituting the double holes by small external handles.

FIG. 5 illustrates a perspective view of prosthesis for multiple grafts, flangeless, with double holes, transfixing, in the same direction of the tubular member, located in the upper, intermediary and lower ends of the tubular member.

FIG. 5 A illustrates a prosthesis that differs from prosthesis of FIG. 5 only by substituting the double holes in the same locations, by small external handles, in the tubular member.

FIG. 6 illustrates a perspective view of prosthesis in clover, flangeless, with double holes in the same direction of the tubular portion, located in the upper, intermediary and lower ends of the tubular member.

FIG. 6 A illustrates a prosthesis that differs from prosthesis of FIG. 6 only by substituting the double holes by small external handles, in the same locations.

FIG. 7 illustrates a perspective view of triangular prosthesis with round edges, flangeless, with double holes in the same direction of the tubular member, located in the upper, intermediary and lower ends of the tubular member.

FIG. 7 A illustrates a prosthesis that differs from prosthesis of FIG. 7 only by substituting the double holes by small external handles, in the same locations.

### DESCRIPTION OF THE PREFERRED EMBODIMENTS

With reference to the detailed description of drawings, the figures illustrate some forms of embodiment of the present invention, in the form of a prosthesis for flangeless anastomosis.

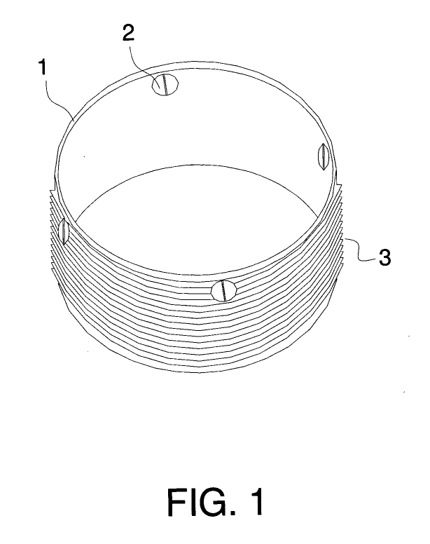

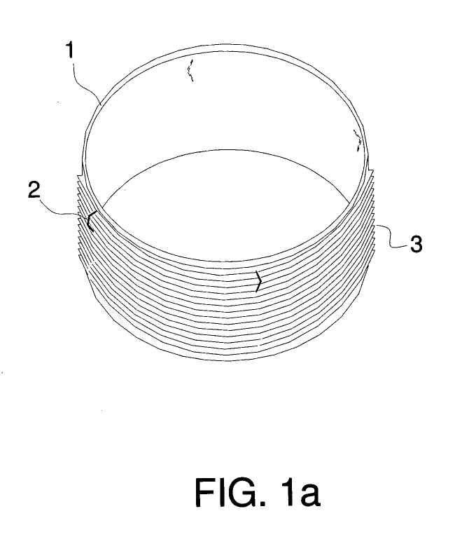

FIG. 1 illustrates a flangeless prosthesis, with tubular member 1 comprising external grooves 3 to anchor the everted grafts, and double holes 2 located in the upper end of prosthesis. These double holes 2 allow the previous fixation of wires that will fixate the prosthesis to the organ wall, facilitating the applicability of the prosthesis, as well as definitely excluding the possibility of intersection of the prosthesis light by wire handles. Also avoiding the rupture of wires when tying-up. In prosthesis comprising flange, the wires, when not previously tied, due to frictions of material of prosthesis, from handles located in the upper surface of the flange, can be ruptured when tying-up. It also reduces remarkably the preparation time of anastomosis, because it uses the same wire that can be passed through the organ wall, contiguous, simultaneously. A very important aspect is related to double holes 2 being in parallel with the tubular member, and perpendicular to the organ wall, the applied traction will be in this direction, also everting the incision edge. After tying-up the wires that fix the prosthesis to the organ wall, it can also make a circumferential external fixation of the incision edge, in the tubular member 1, once the incision is made with a edge turned up. This is extremely important because in no hypothesis there is a protuberance of the anastomotic aggregate (prosthesis-grafts) in the light of the organ, besides assuring a hermetically closed anastomosis. Other aspect of fundamental importance is the inexistence of risk of graft or grafts kinking when emerging from the prosthesis, once the circumferential fixation is distant from this place, allowing the grafts to be free and released in the route inside the prosthesis. Also, the gauge of anastomosis will remain constant from the start to the end of this procedure, without the occurrence of a ‘purse string’ phenomenon that normally happens in conventional anastomosis, leading to an immediate reduction of this gauge. This prosthesis also allows single or multiple anastomosis, at a single time, in the desired quantity of grafts for anastomosis. In this last case, it is necessary a preparation of an anastomotic trunk, in desired extension and gauge, formed by the body joint or just by ends of several grafts, by suture, that can be seromuscular (extraluminal), or by any safe method. The external gauge of the trunk must be equal or slightly superior compared to the prosthesis internal gauge, to be everted to cover the intraluminal portion of prosthesis, externally. One technique for its application could be described as follows: the wires are previously tied-up to double holes 2, the handles are externally to the light of prosthesis. Preferably, these wires must be of different colors, intertwined. This can facilitate the identification of the correct handles to be tied among them. After that, the graft or anastomotic trunk passes through the light of prosthesis, it is everted and fixed, in the exterior, circumferentially, with circular point, anchored in external grooves 3 of prosthesis, avoiding sliding. The anastomotic formed by prosthesis-grafts aggregate and previously tied-up wires of prosthesis is mounted. The place for anastomosis is selected, determining the size of incision, if it is a straight line, its length must be equal to half of external perimeter of prosthesis the tubular member, the wires pass in the edge where the incision is made, equidistant. If four wires of different colors are passed, always the contiguous handles of different color must be tied-up, never of the same color, or same wire (because they are juxtaposed, or interposed by other wire). After passing the wires, the incision is made, digitally tamponed, introducing the anastomotic aggregate, and tying the wires. As said, the wires will traction the organ wall and its edge upward and against the external surface of tubular member 1. When desired, as said, a tie can also be made with a handle of any wire, around the anastomosis, fixing the edge of incision to the tubular member circumferentially, closing it hermetically. Other technical embodiment could be described as follows: the wires are previously tied-up externally to double holes 2; if the graft or trunk is free, it is fixed to the organ wall with opposed simple points located in two ends formed by half of its perimeter (by compressing the end of graft letting it in a straight line and by fixing the tow extreme points). Other simple point is also applied between the graft and organ wall, between two frontal. These three points are tied-up as soon as applied. A fourth point diametrally opposed to the last is applied in other edge of the graft mouth, that is still free, and is not tied-up leaving the long handle, to let the place of straight line incision be visible. The isolated graft or trunk, by its free end, backwardly passes through the light of prosthesis with compatible gauge. The wires previously tied-up to prosthesis, are passed through the following route: they enter in the graft that passed through, inwardly, exactly in the middle of two adjacent points that were applied to fix it to the organ wall. It goes and transfixes the organ wall, or is applied in seromuscular manner, if desired. The two handles of the same wire are applied contiguously, being together. The other wires are applied in the same manner, resulting, if four wires, two by two, opposed an in parallel. Note that the fixation wires of the prosthesis to graft wall, pass in the middle of contiguous wires, applied to previously and partially fixate the graft to the organ wall. The prosthesis goes next to the incision point (note that the prosthesis was not previously fixed to the graft or trunk, it is released with graft passing through its light). There is an incision of the organ wall, digitally tamponed, the wires are tractioned, the prosthesis and graft are introduced to the light of the organ, the wires are tied-up, including the last wire that fixes the graft or trunk edge of the edge in the incision. At the same time, the wires are tractioned and tied-up, the graft or trunk edge is everted and coating the intraluminal portion of the prosthesis, and while tractioning the incision edge upward and fixing externally and circumferentially the external surface of the anastomotic aggregate. Here, if desired, it can be tied with a handle of any fixation wires of prosthesis, fixing the incision edge, externally and circumferentially to anastomotic aggregate.

FIG. 1 A differs from prosthesis of FIG. 1 only by substituting the double holes 2 by small external handles, in the same locations. The role of small handles 2 is the same of the double holes: place for previous fixation of wires that will fix the prosthesis to graft wall. The remaining can be exactly equal, or other technical embodiments can be used.

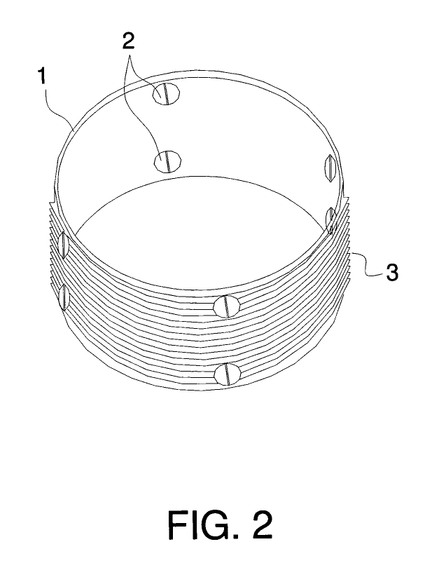

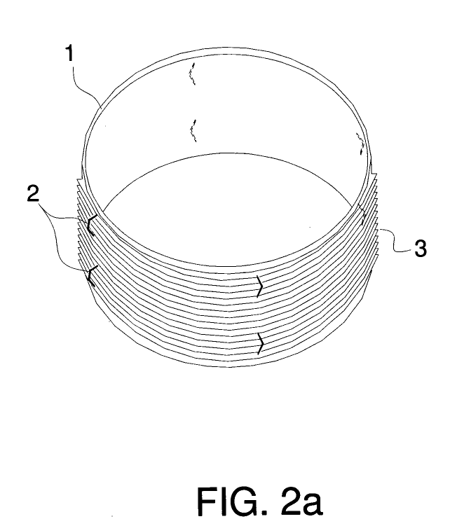

A FIG. 2 e 2 A illustrate prosthesis with fixation resources for wires, double holes and/or handles 2, disposed in the lower edge of tubular member 1. The description serves to both. The double holes or lower handles 2 has several objectives: make the prosthesis that is not bisealed, more versatile by not having upper or inferior end, can be applied in any position; can also fix grafts in lower position, by simple points that pass through them and adventicia or external layer of grafts, avoiding intususception of grafts by the light of prosthesis (to do this, it could also make a fixation in upper holes or handles, however in this last case, it poses the risk of graft kinking, eliminating one of the most important features of these anastomosis, that is the not kinking of grafts independently to where they are positioned, due to the fact they are free in their route through the light of prosthesis); this last fixation, besides avoiding intususception and keeping the characteristic of not kinking of grafts, also facilitating the eversion of graft to cover the prosthesis, besides assuring even more the fixation that can be made with simple points passed by upper holes or by a circular point anchored in external grooves 3 of tubular member 1; if the anastomosis is made in normal wall organs, where the fast clamping could be applied, and the incision can be made before the application of the points, and these applied by lower holes or handles, to a distance from incision edge to be equal or more than the extension of the tubular member, there will never be protuberance of anastomotic aggregate in the light of organ, once the wall is fractioned to the lower edge of anastomotic aggregate, and its exceeding portion will be everted, being with the mouth turned upward, circumferentially to the tubular member, it can be fixed with a circular point, assuring the hermetical feature of anastomosis. However, it also happens even without clamping and previous incision.

The arrangement of holes or small lower handles in relation to the upper, can have the same alignment or intertwined at half distance, being not longitudinally aligned.

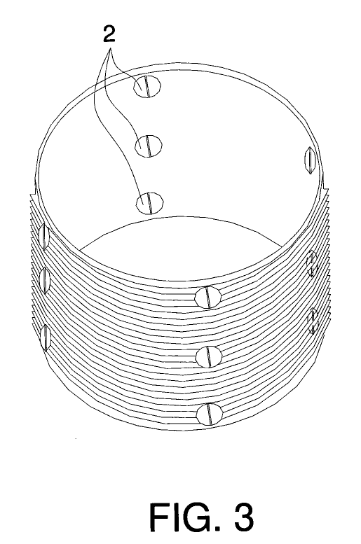

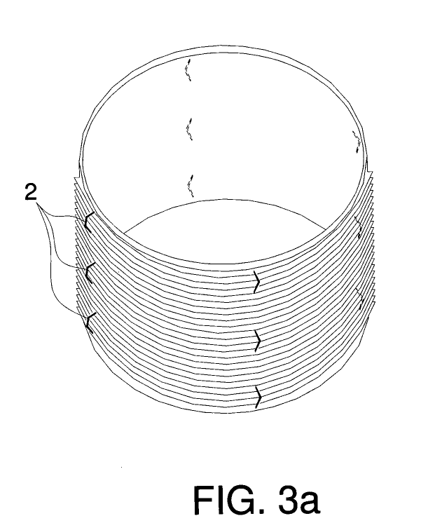

FIGS. 3 and 3 A illustrate prosthesis with double holes or small handles 2 longitudinally aligned in the tubular member. It can also be arranged intertwined, not aligned longitudinally, as mentioned above. These multiple holes or external handles 2 have the same purposes described above, mainly of adjusting the extension of prosthesis that will be introduced in the light of organ, if depending of the wall thickness. Thus, organs with thick walls where pass the fixation wires of prosthesis by upper holes or handles; medium walls, by intermediary and thin walls, by lower holes. Also, as said, even if the wires pass through the upper holes or handles, and if the graft is fixed by the external layer, to the lower, facilitate the eversion to cover the prosthesis, assuring even more the fixation, assuring the not kinking of grafts when emerging from prosthesis, besides avoiding intususception, through the light of prosthesis (for example, in intestines, by peristaltism). It is important noting that prosthesis can be previously related or as fixed organ or outdoor, in any position. Thus, the importance of fixing in the lower portion of the prosthesis, the organs with peristaltism which waves go to meet it, if it passes through the light and are everted to cover it. So, avoiding its intususception by light of prosthesis that could be avoided just by changing the position of the prosthesis, placing it previously in the other organ. Then, the peristaltic waves go away from prosthesis.

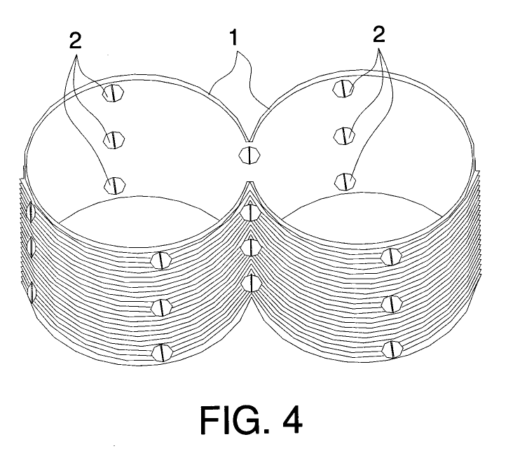

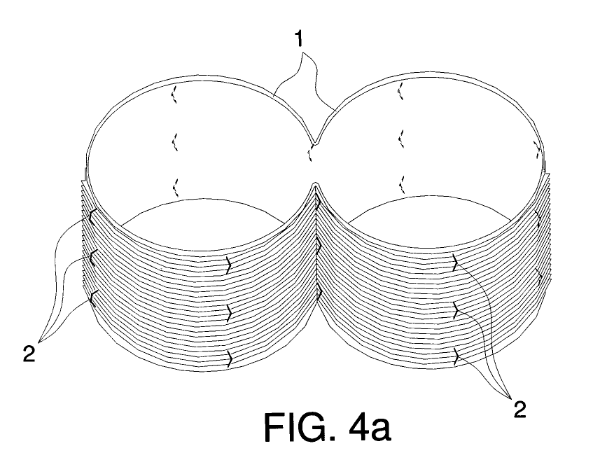

FIGS. 4 and 4 A illustrate prosthesis with multiple double holes or small external handles 2 to tubular member 1. In this specific case, two or more grafts would be previously joined by one of the ends, forming an anastomotic trunk of compatible gauge, that pass through the light of prosthesis, would be everted and fixed, with simple point or other method, by double holes or handles to tubular member, once a circular point can not be applied here to fixate it. Thus, the double holes and handles, besides the prior mentioned functions are also the fixation points of grafts or trunk to prosthesis. The form of these prosthesis can be meaningful to generate a laminal flow, once it preserves the natural form, circular, of grafts that pass through its light.

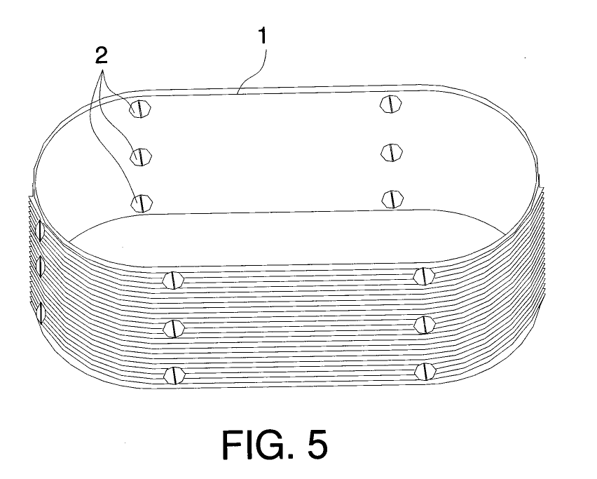

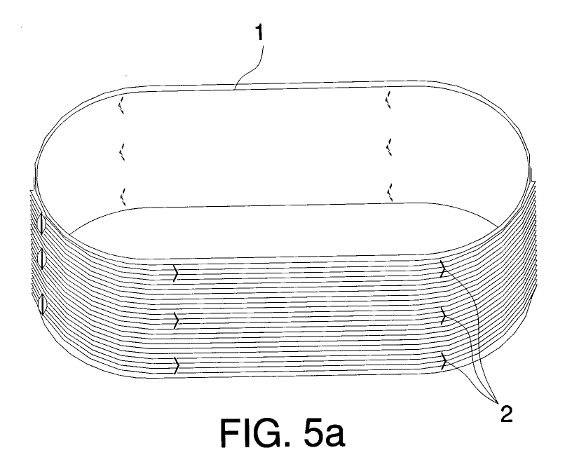

FIG. 5 e 5 A illustrate prosthesis that could accommodate several joined grafts in serial by one of its ends, of equal or different gauges, equal or smaller than the smaller diameter of prosthesis, to assure its original form, without constriction, when individualized. Equally, after everted, fixed to prosthesis by simple points, or other method, by double holes or small external handles 2. Also here, according to the thickness of organ wall that it will receive, the lower, intermediary or upper holes or handles to be chosen to pass the points that will fixate the prosthesis, adjacent wall of the organ submitted to incision.

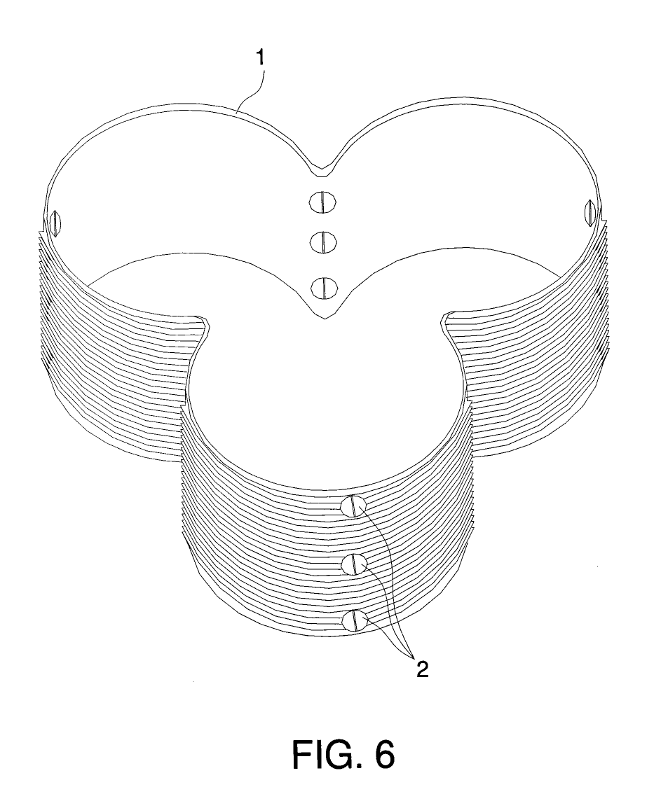

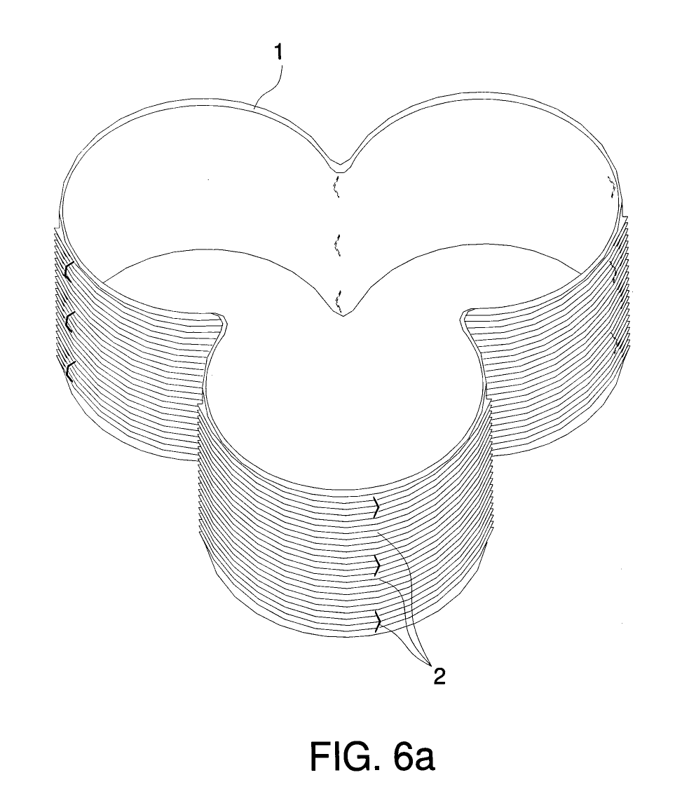

FIG. 6 e 6 A illustrate prosthesis also with double holes and/or upper, intermediary or upper handles 2 (the same prosthesis can comprise double holes and handles). In the same manner, it can accommodate several joined grafts in an anastomotic trunk by one of its ends, keeping the original diameter in its origin. Maybe, this can be important from the physiological point of view for anastomosis, in the generation of laminal flow since its origin. Here, because it is not possible to apply the circumferential point for the fixation of grafts, the handles and double holes 2 are also used to its purpose.

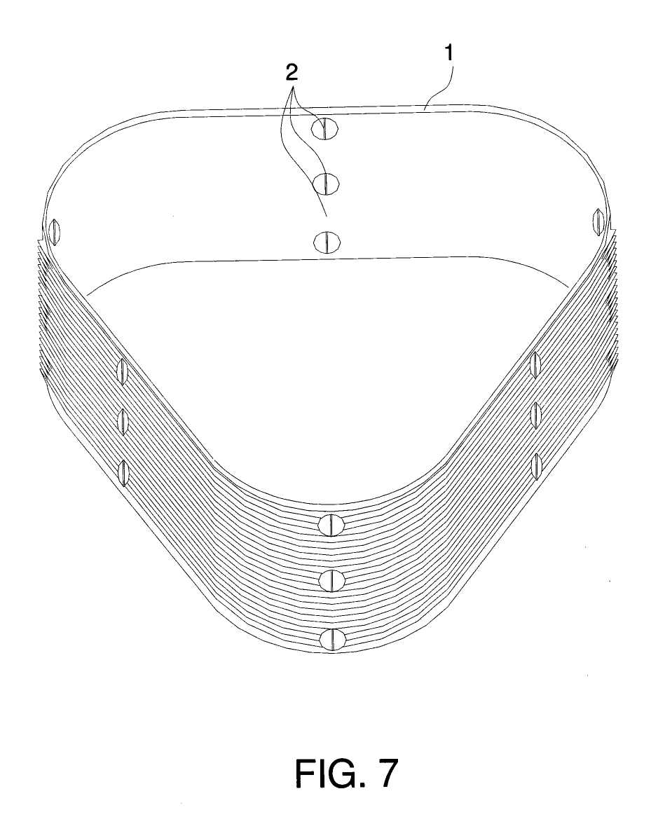

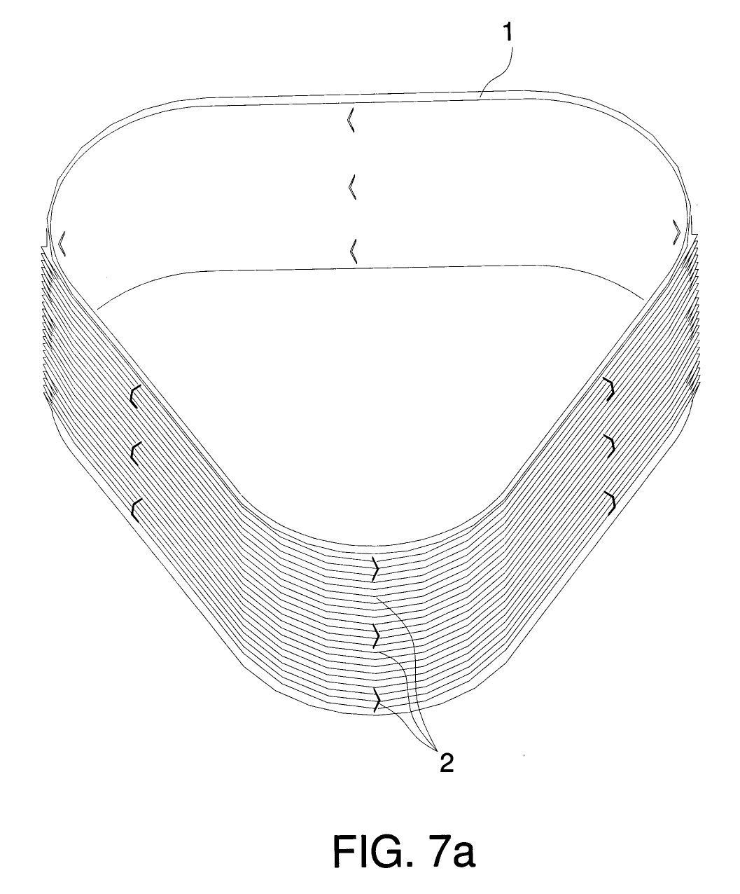

FIGS. 7 and 7 A illustrate prosthesis in clover also has double holes and/or small external handles 2 to tubular member 1 with the same function already described. It can also accommodate multiple grafts joined by any method by one of its ends, in anastomotic trunk.

With this prosthesis, single or multiple anastomosis can be prepared, at a single time, wide, without clamping and sutureless, or if preferred, in normal wall organs, with expeditious clamping and sutureless, of any type end-to-end, side-to-side or end-to-side, between any tubular organs.

1. Flangeless prosthesis for anastomosis comprising: a tubular member and a lumen, wherein the tubular member (1) has varied shapes and size at least one double hole or an external handle (2) in an end portion thereof.

2. Flangeless prosthesis, according to claim 1, wherein the at least one double hole or external handle (2) are located on an upper end of the tubular member.

3. Flangeless prosthesis, according to claim 1, wherein the at least one double hole or external handle (2) are located in an upper or lower end of the tubular member (1).

4. Flangeless prosthesis, according to claim 1, wherein the at least one double hole or external handle (2) are located in upper intermediary or lower portion in the tubular member (1), longitudinally aligned or not.

5. Flangeless prosthesis, according to claim 1, wherein the at least one double hole or external handle (2) regulate extension of an intraluminal portion, avoiding its protuberance into the lumen of the organ.

6. Flangeless prosthesis, according to claim 1, wherein a fixation of grafts in the at least one double hole or external handle (2) avoids intussusception of grafts.

7. Flangeless prosthesis, according to claim 1, wherein the tubular member (1) comprises grooves (3) in the external surface of the side wall, in a transversal plan in direction to the lumen of the tubular member.
